(19) United States
(12) Patent Application Publication
(10) Pub. No.: US 2009/0306170 A1
(43) Pub. Date: Dec. 10, 2009

(54) SYNTHESIS OF TEGASEROD OR A SALT THEREOF

(75) Inventors: Abhay Gaitonde, Maharashtra (IN); Bindu Mnaojkumar, Maharashtra (IN); Sandeep Sonawane, Maharashtra (IN)

Correspondence Address: FULBRIGHT & JAWORSKI L.L.P.;Attn: MN IP Docket, 600 Congress Avenue, Suite 2400, Austin, TX, 78701, US

(73) Assignee: GENERICS UK LIMITED, HERTFORDSHIRE (GB)

(21) Appl. No.: 12/513,800

(22) PCT Filed: Nov. 9, 2007
(86) PCT No.: PCT/EP07/62176

§ 371 Date: Jul. 8, 2009

(30) Foreign Application Priority Data
Nov. 9, 2006 (IN) 1857/MUM/2006

(51) Int. Cl. A61K 31/404; C07D 209/14; A61P 1/00

(52) U.S. Cl. 514/415; 548/505

(57) ABSTRACT

The present invention relates to a novel process for the synthesis of 1-((5-methoxy-1H-indol-3-yl)methyleneamino)-3-pentyl-guanidine, commonly known as tegaserod, which is used as a gastroprokinetic, and salts thereof. The present invention also relates to tegaserod and salts thereof having an HPLC purity of about 95% or more. The present invention further relates to pharmaceutical compositions comprising tegaserod or a salt thereof, second medical uses of tegaserod or a salt thereof, and methods of treating or preventing irritable bowel syndrome using tegaserod or a salt thereof.

### CROSS-REFERENCE TO RELATED APPLICATION(S)

This application is a Section 371 National Stage Application of International No. PCT/EP2007/062176, filed 9 Nov. 2007 and published as WO 2008/055994 A1 on 15 May 2008, which claims priority from the India Application 1857/MUM/2006, filed 9 Nov. 2006, the contents of which are incorporated herein in their entirety for all purposes.

### FIELD OF THE INVENTION

The present invention relates to a novel process for the synthesis of 1-((5-methoxy-1H-indol-3-yl)methyleneamino)-3-pentyl-guanidine, commonly known as tegaserod, which is used as a gastroprokinetic, and salts thereof. The present invention also relates to tegaserod and salts thereof having an HPLC purity of about 95% or more. The present invention further relates to pharmaceutical compositions comprising tegaserod or a salt thereof, second medical uses of tegaserod or a salt thereof, and methods of treating or preventing irritable bowel syndrome using tegaserod or a salt thereof.

### BACKGROUND OF THE INVENTION

Tegaserod, shown below, represents a new class of drugs (aminoguanidine indoles) and is a partial 5-HT₄ receptor agonist. Tegaserod is used for the management of constipation-predominant irritable bowel syndrome (IBS).

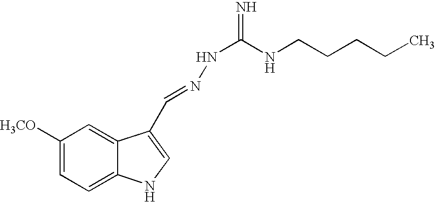

U.S. Pat. No. 5,510,353 first described tegaserod and its synthetic route. This patent reports the coupling of 5-methoxy-indole-3-carboxaldehyde and N-pentyl-N′-amino-guanidine hydroiodide in methanol using conc. HCl. The coupling reaction gives an impure product that necessitates column chromatography. The process reported in U.S. Pat. No. 5,510,353 uses column chromatography to isolate tegaserod free base. Further, N-pentyl-N′-amino-guanidine hydroiodide used in this process is prohibitively expensive.

WO 2005/105740 discloses a process for the preparation of tegaserod and its maleate salt. This patent application reports the coupling of 5-methoxy-indole-3-carboxaldehyde and N-pentyl-N′-amino-guanidine hydroiodide in water in the presence of organic/inorganic acids or organic/inorganic bases. It is further disclosed in WO 2005/105740 that coupling of 5-methoxy-indole-3-carboxaldehyde and N-pentyl-N′-amino-guanidine hydroiodide gives a better purity, when the reaction is carried out in water (purity of crude product=94.02 using triethylamine as base, purity of crude product=91.55 using sodium bicarbonate as base).

Buchheit et al. (Journal of Medicinal Chemistry, 1995, vol. 38, no. 13, pages 2331-2338) disclose a process for the preparation of N-pentyl-N′-amino-guanidine hydroiodide. The purity of N-pentyl-N′-amino-guanidine hydroiodide as prepared by this process is very low.

Therefore, a need exits for a process that overcomes one of more of the disadvantages of the current processes.

### SUMMARY OF THE INVENTION

The difficulties encountered in the prior art for the preparation of tegaserod have been successfully overcome in the present invention. The process of the present invention provides a pure product that can be used without column chromatography. Thus the present invention is more suited to scale-up. Further, the process of the present invention is economically viable, since it avoids the use of N-pentyl-N′-amino-guanidine hydroiodide. Finally, in the present invention all the intermediates are solids and therefore purification can be done by simple crystallization processes.

A first aspect of the present invention provides a process of preparing tegaserod or a salt thereof, wherein the process does not comprise the use of N-pentyl-N′-amino-guanidine or a salt thereof.

In a preferred embodiment of the first aspect of the present invention, the process of preparing tegaserod or a salt thereof comprises the steps of:
(a) coupling S-methyl-isothiosemicarbazide or a salt thereof and 5-methoxy-indole-3-carboxaldehyde to form 1-((5-methoxy-1H-indol-3-yl)methylene)-S-methyl-isothiosemicarbazide:

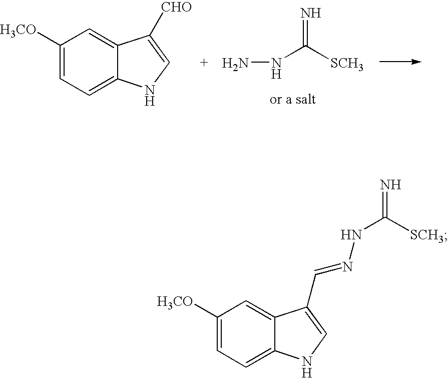

and
(b) reacting the 1-((5-methoxy-1H-indol-3-yl)methylene)-S-methyl-isothiosemicarbazide with n-pentyl amine to form tegaserod:

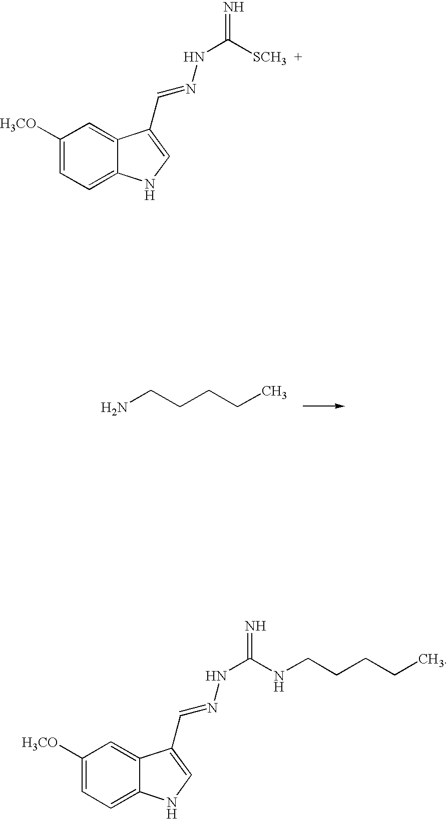

The skilled person will appreciate that:
S-methyl-isothiosemicarbazide and salts thereof exist in two tautomeric forms:

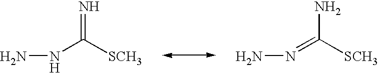

1-((5-methoxy-1H-indol-3-yl)methylene)-S-methyl-isothiosemicarbazide exists in four tautomeric forms:

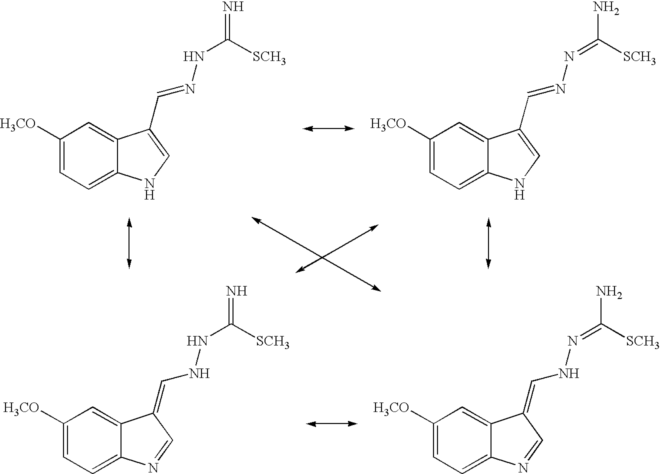

tegaserod exists in four tautomeric forms:

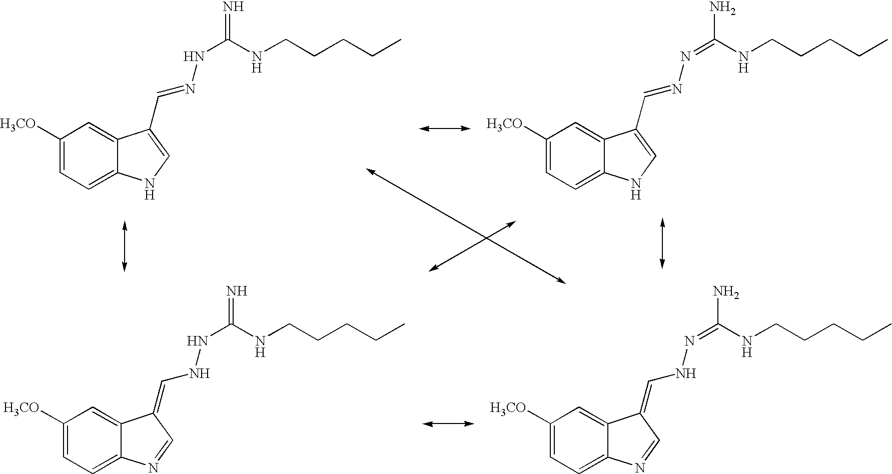

It is to be understood that where tautomeric forms occur, the present invention embraces all tautomeric forms and their mixtures, i.e. although S-methyl-isothio-semicarbazide and 1-((5-methoxy-1H-indol-3-yl)methylene)-S-methyl-isothiosemi-carbazide are mostly defined for convenience by reference to one isothiosemicarbazide form only, and although tegaserod is mostly defined for convenience by reference to one guanidino form only, the invention is not to be understood as being in any way limited by the particular nomenclature or graphical representation employed.

When an S-methyl-isothiosemicarbazide salt is used in the process of the present invention, this may be an acid addition salt with acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid), or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, p-toluenesulfonic, naphthalene-2-sulfonic or camphorsulfonic acid) or amino acids (for example, ornithinic, glutamic or aspartic acid). Preferably the S-methyl-isothiosemicarbazide salt is a hydrohalide (such as the hydrofluoride, hydrochloride, hydrobromide, or hydroiodide) or a sulfonate (such as the methanesulfonate, benzenesulfonate, or p-toluenesulfonate). Preferably the S-methyl-isothiosemicarbazide salt is S-methyl-isothiosemicarbazide hydroiodide.

Preferably step (a) is carried out in the presence of a base, in particular if a S-methyl-isothiosemicarbazide salt is used. The base may be an organic or inorganic base. Suitable organic bases are C₃-C₈ tertiary amines, such as triethylamine. Suitable inorganic bases are sodium hydroxide, sodium bicarbonate, potassium carbonate, or sodium carbonate.

Preferably step (a) and/or step (b) are carried out in an organic solvent. The organic solvent may be a C₁-C₈ alcohol, acetonitrile, a C₂-C₈ ether, or a C₃-C₈ ester. A preferred C₁-C₈ alcohol is methanol.

Preferably the tegaserod or the salt thereof is obtained on an industrial scale. This means that the tegaserod or the salt thereof is preferably obtained in batches of 0.5 kg, 1 kg, 5 kg, 10 kg, 50 kg, 100 kg, 500 kg or more.

Preferably the HPLC purity of the tegaserod obtained is about 95% or more, preferably about 96% or more, preferably about 97% or more, preferably about 98% or more.

The tegaserod may further be converted into a tegaserod salt, such as tegaserod maleate. Preferably the HPLC purity of the tegaserod salt obtained is about 95% or more, preferably about 96% or more, preferably about 97% or more, preferably about 98% or more, preferably about 99% or more, preferably about 99.5% or more.

A second aspect of the present invention provides tegaserod or a salt thereof, obtained by a process according to the first aspect of the present invention. Preferably the tegaserod or salt thereof is suitable for use in medicine, preferably for treating or preventing irritable bowel syndrome.

A third aspect of the present invention provides a pharmaceutical composition comprising the tegaserod or salt thereof according to the second aspect of the present invention, and one or more pharmaceutically acceptable excipients or diluents.

A fourth aspect of the present invention provides a use of the tegaserod or salt thereof according to the second aspect of the present invention for the manufacture of a medicament for the treatment or prevention of irritable bowel syndrome.

A fifth aspect of the present invention provides a method of treating or preventing irritable bowel syndrome, comprising administering a therapeutically or prophylactically effective amount of the tegaserod or salt thereof according to the second aspect of the present invention to a patient in need thereof. Preferably the patient is a mammal, preferably a human. Preferably the amount of the tegaserod or salt thereof administered is from 0.1 mg to 50 mg per kg per day.

### DETAILED DESCRIPTION OF THE INVENTION

Tegaserod, used for the treatment of irritable bowel syndrome, can be manufactured in high purity by the process of the present invention. A preferred embodiment of the present invention involves a coupling reaction between S-methyl-isothio-semicarbazide hydroiodide with 5-methoxy-indole-3-carboxaldehyde in an organic solvent in the presence of a base as the first step. The product of this step is coupled with n-pentyl amine in an organic solvent to yield tegaserod in high purity. S-Methyl-isothiosemicarbazide salts other than the hydroiodide can be used, for example, other hydrohalides, sulfonates etc. A preferred salt is the hydroiodide salt. The present invention avoids the use of N-pentyl-N′-amino-guanidine hydroiodide. The present invention is thus a novel two stage approach to tegaserod, that offers better control over the purity of the drug substance and a significant cost advantage compared to processes described in the prior art.

The present invention yields tegaserod with a purity of around 95%.

A further embodiment of the present invention also comprises compositions of tegaserod of high purity along with pharmaceutically acceptable excipients.

The following synthetic scheme demonstrates a preferred process of the present invention.

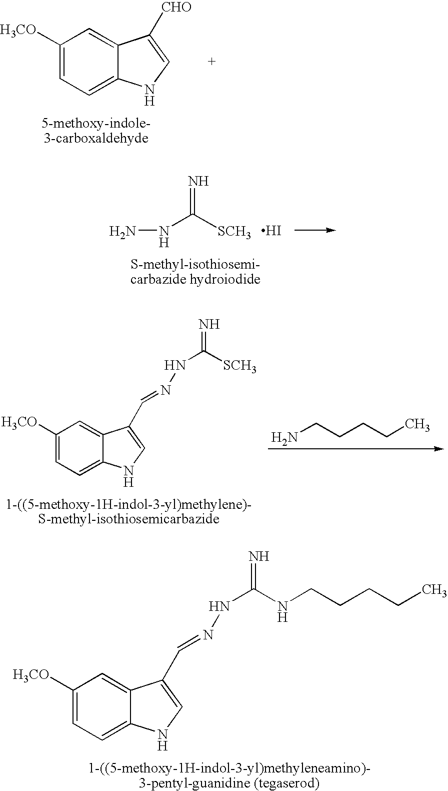

The invention is now demonstrated by the following non-limiting illustrative example.

### EXAMPLE

### Step 1: Schiff's Base Formation of 5-methoxy-indole-3-carboxaldehyde and S-methyl-isothiosemi-carbazide hydroiodide

5-Methoxy-indole-3-carboxaldehyde (1.5 g, 1 eq) and S-methyl-isothiosemicarbazide hydroiodide (3.99 g, 2 eq) in methanol (15 ml, 10 vol) were stirred in the presence of triethylamine (3 ml, 2 vol) at 25-30° C. for 2 hours. After completion of the reaction, the methanol was removed by distillation under reduced pressure at 45-50° C. and ethyl acetate (10.5 ml, 7 vol) was added to the residue to precipitate out the product. The product, 1-((5-methoxy-1H-indol-3-yl)methylene)-S-methyl-isothiosemi-carbazide, was separated by filtration, washed with ethyl acetate (3 ml, 2 vol) and dried under vacuum at 45-50° C. The yield was almost quantitative (˜100%).

### Step 2: Conversion of 1-((5-methoxy-1H-indol-3-yl)methylene)-S-methyl-isothiosemicarbazide to 1-((5-methoxy-1H-indol-3-yl)methyleneamino)-3-pentyl-guanidine (Tegaserod)

A solution of 1-((5-methoxy-1H-indol-3-yl)methylene)-S-methyl-isothiosemicarbazide (8.0 g, 1 eq) and n-pentyl amine (2.65 g, 1 eq) was refluxed in methanol (8 ml, 1 vol) at 66° C. for 4 hours. After completion of the reaction, the methanol was removed by distillation under reduced pressure at 45-50° C. to obtain tegaserod free base as a yellowish brown solid. Yield=97%. HPLC purity=95%.

### Step 3: Conversion of 1-((5-methoxy-1H-indol-3-yl)methyleneamino)-3-pentyl-guanidine (Tegaserod) to Tegaserod Maleate

1-((5-Methoxy-1H-indol-3-yl)methyleneamino)-3-pentyl-guanidine (55 g, 1 eq) was taken in methanol (357.5 ml, 6.5 vol) and stirred. To this reaction mixture was added at room temperature a solution of maleic acid (74.15 g, 3.5 eq) in water (137.5 ml, 2.5 vol) and the reaction mixture stirred for one hour at room temperature. The solid obtained was then filtered through a Buchner funnel and dried at 700 mmHg and 500° C. Yield=36.8 g, 48.42%. HPLC purity=99.45%.

What is claimed is:

1. A process of preparing tegaserod or a salt thereof, comprising the steps of:
(a) coupling S-methyl-isothiosemicarbazide or a salt thereof and 5-methoxy-indole-3-carboxaldehyde to form 1-((5-methoxy-1H-indol-3-yl)methylene)-S-methyl-isothiosemicarbazide; and
(b) reacting the 1-((5-methoxy-1H-indol-3-yl)methylene)-S-methyl-isothiosemicarbazide with n-pentyl amine to form tegaserod.

2. The process as claimed in claim 1, wherein the S-methyl-isothiosemicarbazide salt is a hydrohalide, a sulfonate or a mixture thereof.

3. The process as claimed in claim 2, wherein the hydrohalide is hydroiodide or wherein the sulfonate is methanesulfonate, benzenesulfonate or p-toluenesulfonate.

4. The process as claimed in claim 1, wherein step (a) is carried out in the presence of a base.

5. The process as claimed in claim 4, wherein the base is an organic or inorganic base.

6. The process as claimed in claim 5, wherein the organic base is a C₃-C₈ tertiary amine.

7. The process as claimed in claim 6, wherein the organic base is triethylamine.

8. The process as claimed in claim 5, wherein the inorganic base is sodium hydroxide, sodium bicarbonate, potassium carbonate, sodium carbonate or a mixture thereof.

9. The process as claimed in claim 1, wherein step (a) or step (b) or both steps (a) and (b) are carried out in an organic solvent.

10. The process as claimed in claim 9, wherein the organic solvent is a C₁-C₈ alcohol, acetonitrile, a C₂-C₈ ether, a C₃-C₈ ester or a mixture thereof.

11. The process as claimed in claim 10, wherein the organic solvent is methanol.

12. The process as claimed in claim 1, wherein the tegaserod or the salt thereof is obtained in batches of 0.5 kg or more.

13. The process as claimed in claim 1, wherein the HPLC purity of the tegaserod obtained is about 95% or more.

14. The process as claimed in claim 1, wherein the tegaserod is further converted into a tegaserod salt.

15. The process as claimed in claim 14, wherein the tegaserod salt is tegaserod maleate.

16. The process as claimed in claim 14, wherein the HPLC purity of the tegaserod salt obtained is about 95% or more.

17. Tegaserod or a salt thereof, obtained by a process as claimed in claim 1.

18. A pharmaceutical composition comprising the tegaserod or salt thereof as claimed in claim 17 and a carrier.

19. A method of treating or preventing irritable bowel syndrome, comprising administering a therapeutically or prophylactically effective amount of the tegaserod or salt thereof as claimed in claim 17 to a patient in need thereof.

20. The method as claimed in claim 19, wherein the patient is a mammal.

21. The method as claimed in claim 20, wherein the patient is a human.

22. The method as claimed in claim 19, wherein the amount of the tegaserod or salt thereof administered is from 0.1 mg to 50 mg per kg per day.

23. A process of preparing tegaserod or a salt thereof, wherein the process does not comprise the use of N-pentyl-N′-amino-guanidine or a salt thereof.

24. The process as claimed claim 23, wherein the tegaserod or the salt thereof is obtained in batches of 0.5 kg or more.

25. The process as claimed claim 23, wherein the HPLC purity of the tegaserod obtained is about 95% or more.

26. The process as claimed claim 23, wherein the tegaserod is further converted into a tegaserod salt.

27. The process as claimed in claim 26, wherein the tegaserod salt is tegaserod maleate.

28. The process as claimed in claim 26, wherein the HPLC purity of the tegaserod salt obtained is about 95% or more.

29. Tegaserod or a salt thereof, obtained by a process as claimed in claim 23.

30. A pharmaceutical composition comprising the tegaserod or salt thereof as claimed in claim 29 and a carrier.

31. A method of treating or preventing irritable bowel syndrome, comprising administering a therapeutically or prophylactically effective amount of the tegaserod or salt thereof as claimed in claim 29 to a patient in need thereof.

32. The method as claimed in claim 31, wherein the patient is a mammal.

33. The method as claimed in claim 32, wherein the patient is a human.

34. The method as claimed in claim 31, wherein the amount of the tegaserod or salt thereof administered is from 0.1 mg to 50 mg per kg per day.
